(19) United States
(12) Patent Application Publication
(10) Pub. No.: US 2013/0138196 A1
(43) Pub. Date: May 30, 2013

(54) ELECTRODE PAD SET

(71) Applicants: Cheng-Lun TSAI, Taoyuan County (TW); Kang Ping Lin, Taoyuan County (TW); Yi-Rou Lin, Yunlin County (TW); Yu-Ju Chiang, Yilan City (TW); Shih-Wei Liu, Taichung City (TW)

(72) Inventors: Cheng-Lun TSAI, Taoyuan County (TW); Kang Ping Lin, Taoyuan County (TW); Yi-Rou Lin, Yunlin County (TW); Yu-Ju Chiang, Yilan City (TW); Shih-Wei Liu, Taichung City (TW)

(21) Appl. No.: 13/306,844

(22) Filed: Nov. 29, 2011

(51) Int. Cl. A61N 1/04

(52) U.S. Cl. 607/152

(57) ABSTRACT

An electrode pad set comprises a left electrode pad, a right electrode pad, a first indication figure on the left electrode pad, a second indication figure on the right electrode pad. The left electrode pad and the right electrode pad respectively have a first warning sign with a first shape and a second warning sign with a second shape corresponding to the first shape. The perimeter of the first warning sign has a first coating with a first color. The perimeter of the second warning sign has a second coating with a second color. A left electrode pad symbol of the first indication figure has a color identical to the first color. A right electrode pad symbol of the second indication figure has a color identical to the second color. The present invention uses colors, shapes and indication figures to promote the success rate of attaching the electrode pads.

### BACKGROUND OF THE INVENTION

1. Field of the Invention

The present invention relates to an electrode pad set, particularly to an error-proof electrode pad set.

2. Description of the Related Art

Electrode pads are widely used in medicine. For example, defibrillation electrode pads are used in a defibrillator to give defibrillation shock. Normally, one defibrillation electrode pad is attached to a region below the collar bone and the right side of chest bone; the other electrode pad is attached to a region outside the left nipple and in the mid-axillary line, whereby the current passing the heart with maximum area. The performance of a defibrillator depends on the positions where the electrode pads are attached and the electric conduction between the electrode pad and the skin. In emergencies, the operators of automated external defibrillators are normally lay persons. In such a case, it is hard to attach the electrode pads to the correct positions. Therefore, the error-proof design of electrode pads is very important.

The electrode pad sets of the defibrillators available in the market normally use colors and schematic diagrams to distinguish the left electrode pad from the right electrode pad. In an experiment undertaken by Anthony D. Andre, et al. in 2004, four brands of defibrillators are randomly distributed to sixteen untrained users. It is found that there are two pad-related factors among the main factors affecting the performance of the defibrillators, which are the electrode pads do not provide the relative attachment positions of the left pad to the right pad and that failed to provide the detailed attachment instruction.

As a result, the present invention proposes of a novel electrode pad set to provide intuitive design for users to effectively overcome the abovementioned problems.

### SUMMARY OF THE INVENTION

The primary objective of the present invention is to provide an electrode pad set, whose left and right electrode pads respectively have different colors, different shapes, and different indication figures showing the positions where the left and right electrode pads should be attached, whereby increase the rate of correctly attaching the electrode pads.

To achieve the abovementioned objective, the present invention proposes an electrode pad set, which comprises a left electrode pad having a first warning sign, a right electrode pad having a second warning sign, a first indication figure on the left electrode pad, and a second indication figure on the right electrode pad. The first warning sign has a first shape, and the second warning sign has a second shape corresponding to the first shape. The perimeter of the first warning sign has a first coating with a first color. A left electrode pad symbol of the first indication figure has a color identical to the first color. The perimeter of the second warning sign has a second coating with a second color. A right electrode pad symbol of the second indication figure has a color identical to the second color.

Below, the embodiments are described in detail in cooperation with the drawings to make easily understood the structural characteristics, technical contents and efficacies of the present invention.

### BRIEF DESCRIPTION OF THE DRAWINGS

FIG. 1 is a diagram schematically showing the folded state of an electrode pad set according to one embodiment of the present invention;

FIG. 2 is a diagram schematically showing the unfolded state of the electrode pad set according to the embodiment shown in FIG. 1; and

FIG. 3 is a diagram schematically showing the unfolded state of an electrode pad set according to another embodiment of the present invention.

### DETAILED DESCRIPTION OF THE INVENTION

The spirit of the novel electrode pad set of the present invention is to use different colors, different shapes and different indication figures to make users able to instinctively attach the electrode pad to the correct positions.

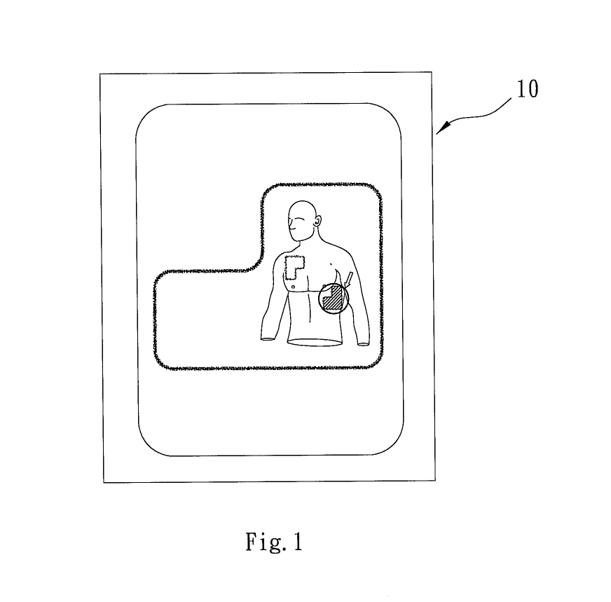

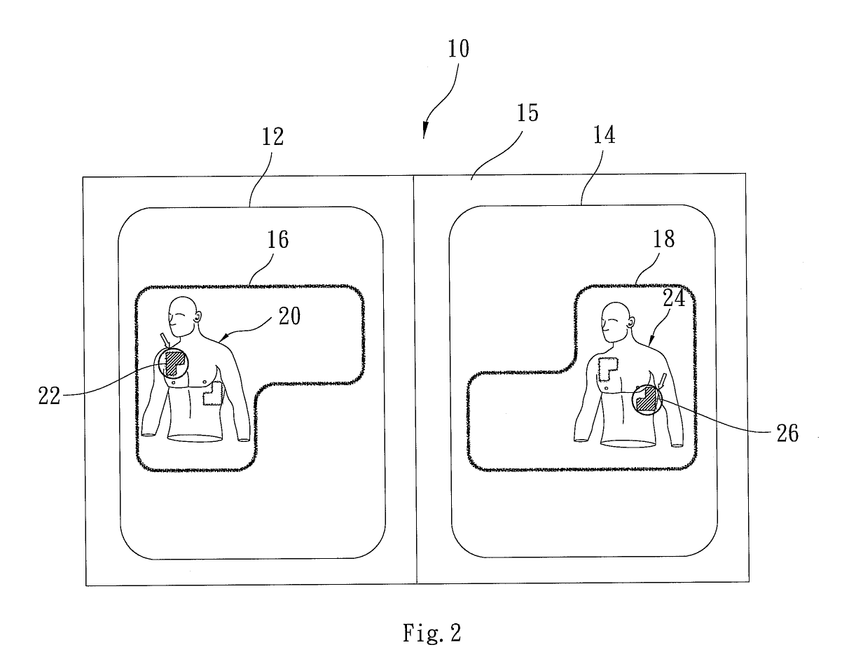

Refer to FIG. 1 and FIG. 2 respectively diagrams schematically showing the folded state and unfolded state of an electrode pad set according to one embodiment of the present invention.

The electrode pad set 10 of the present invention comprises a left electrode pad 12 and a right electrode pad 14 attach to backing adhesives 15. The left electrode pad 12 has a first warning sign 16 with a first shape. The right electrode pad 14 has a second warning sign 18 with a second shape corresponding to the first shape. The perimeter of the first warning sign 16 has a first coating with a first color. The perimeter of the second warning sign 18 has a second coating with a second color. The first color is different from the second color so as to increase the distinguishability of the left and right electrode pads 12 and 14 respectively.

A first indication FIG. 20 indicating the position where the left electrode pad 12 should be attached is printed on the surface of the left electrode pad 12. A left electrode pad symbol 22 on the first indication FIG. 20 has the same color as the first color of the first coating. A second indication FIG. 24 indicating the position where the right electrode pad 14 should be attached is printed on the surface of the right electrode pad 14. A right electrode pad symbol 26 on the second indication FIG. 24 has the same color as the second color of the second coating.

The left electrode pad symbol 22 on the first indication FIG. 20 is encircled by a bold line and indicated by an arrow to enhance the recognizability thereof. Similarly, the right electrode pad symbol 26 on the second indication FIG. 24 is also encircled by a bold line and indicated by an arrow to enhance the recognizability thereof.

For example, the perimeter of the first warning sign 16 has a green coating. The perimeter of the second warning sign 18 has a yellow coating. After the users open the package of the electrode pad set, they can easily learn the positions where the left and right electrode pads 12 and 14 are to be attached via the colors of the coatings and then fast complete the process of attaching the left and right electrode pads 12 and 14.

As mentioned above, the first indication FIG. 20 indicating the position where the left electrode pad 12 is to be attached is printed on the surface of the left electrode pad 12; the second indication FIG. 24 indicating the position where the right electrode pad 14 is to be attached is printed on the surface of the right electrode pad 14. Further, the first and second indication FIGS. 20 and 24 are presented in a perspective way to make the user able to exactly learn the positions where the left and right electrode pads 12 and 14 should be attached. Corresponding to the green perimeter of the first warning sign 16, the perimeter of the left electrode pad symbol 22 of the first indication FIG. 20 is colored with green. The left electrode pad symbol 22 has a filled-up interior and is encircled with a bold line and indicated by an arrow so as to enhance the recognizability thereof. The second indication FIG. 24 is also designed according to the same principle. Thereby, the user can easily and fast learn which electrode pad is corresponding to which electrode pad symbol and thus can attach the left and right electrode pads 12 and 14 to the correct positions.

The left and right warning signs 16 and 18 are a pair of corresponding shapes, and the left and right electrode pad symbols 22 and 26 respectively have the same shapes as the left and right warning signs 16 and 18. For example, the first shape of the first warning sign 16 of the left electrode pad symbol 22 are similar to a L-shaped, and the second shape of the second warning sign 18 of the right electrode pad symbol 26 are similar to a inverted L-shaped, wherein L-shaped is corresponding to inverted L-shaped to form a circular arc frame. Thereby, the user can instinctively attach the left and right electrode pads 12 and 14 to the correct positions.

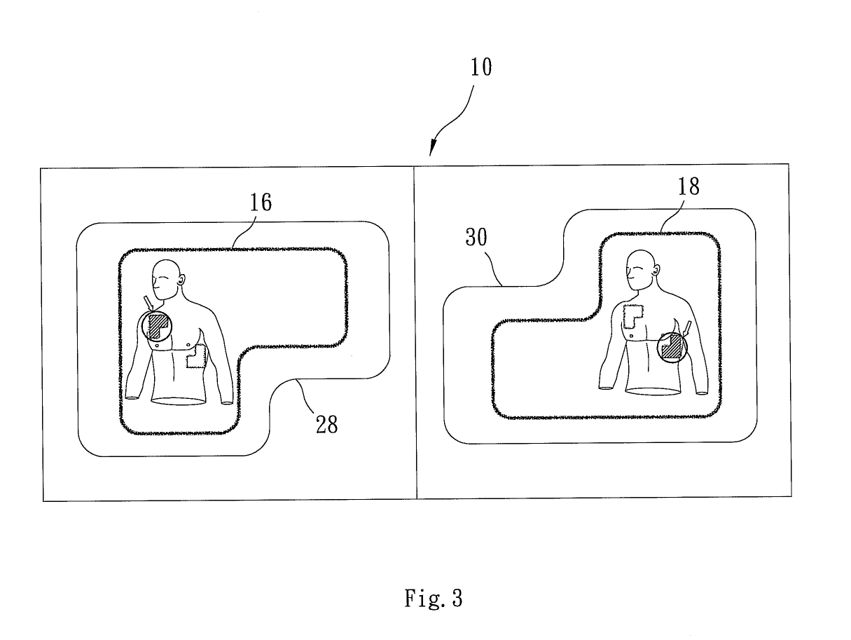

Refer to FIG. 3 for another embodiment of the present invention. In this embodiment, the left and right electrode pads 28 and 30 are fabricated to respectively have the shapes of the left and right warning signs 16 and 18 printed thereon, whereby the user can more instinctively attach the electrode pads to the correct positions. In this embodiment, when the users attach the left or right electrode pad 28 or 30 to the incorrect side of the human body, they will immediately perceive the wrong operation because the shape of the left or right electrode pad 28 or 30 makes it hard to be stuck to the incorrect side.

The embodiments described above are only to exemplify the present invention but not to limit the scope of the present invention. Any equivalent modification or variation according to the spirit of the present invention is to be also included with the scope of the present invention.

What is claimed is:

1. An electrode pad set comprising
a left electrode pad and a right electrode pad, which respectively have a first warning sign with a first shape and a second warning sign with a second shape, wherein said first shape is corresponding to said second shape, and wherein a perimeter of said first warning sign has a first coating with a first color, and wherein a perimeter of said second warning sign has a second coating with a second color;
a first indication figure formed on a surface of said left electrode pad and showing a position where said left electrode pad should be attached, wherein a left electrode pad symbol on said first indication figure has a color identical to said first color of said first coating; and
a second indication figure formed on a surface of said right electrode pad and showing a position where said right electrode pad should be attached, wherein a right electrode pad symbol on said second indication figure has a color identical to said second color of said second coating.

2. The electrode pad set according to claim 1, wherein said first shape of said first warning sign of said left electrode pad is similar to a L-shaped, and wherein said second shape of said second warning sign of said right electrode pad is similar to a inverted L-shaped, wherein said L-shaped is corresponding to said inverted L-shaped to form a circular arc frame.

3. The electrode pad set according to claim 1, wherein said left electrode pad symbol of said first indication figure is encircled by a bold line and indicated by an arrow.

4. The electrode pad set according to claim 1, wherein said right electrode pad symbol of said second indication figure is encircled by a bold line and indicated by an arrow.

5. The electrode pad set according to claim 1, wherein said left electrode pad has a shape identical to said first shape of said first warning sign, and wherein said right electrode pad has a shape identical to said second shape of said second warning sign.
